(19) United States
(12) Patent Application Publication
(10) Pub. No.: US 2015/0320348 A1
(43) Pub. Date: Nov. 12, 2015

(54) DISPOSABLE SAMPLE COLLECTION METHOD AND APPARATUS

(71) Applicant: LIGHTSTAT, LLC, Southlake,, TX (US)

(72) Inventor: Ayal Ram, Ramat Gan (IL)

(21) Appl. No.: 14/443,513

(22) PCT Filed: Nov. 18, 2013
(86) PCT No.: PCT/US2013/070539

§ 371 Date: May 18, 2015

Related U.S. Application Data
(60) Provisional application No. 61/796,648, filed on Nov. 16, 2012.

(51) Int. Cl. A61B 5/15; A61B 5/151; B01L 3/00

(52) U.S. Cl. CPC A61B 5/150412; B01L 3/502715; A61B 5/150755; A61B 5/15113; A61B 5/15117; B01L 2300/0858; B01L 2300/0851; B01L 2300/0672; B01L 2200/027

(57) ABSTRACT

A micro-fluidic device is defined including a channel for conveying blood fluid. A container is defined within the channel for capturing debris generated from the puncture of skin.

### CROSS REFERENCE TO RELATED APPLICATIONS

This application is a 371 national stage of PCT/US2013/070539 filed Nov. 18, 2013, which claims the benefit of U.S. Provisional Application No. 61/796,648 filed Nov. 16, 2012, the entire contents of which are hereby incorporated by reference in their entirety.

### FIELD OF THE INVENTION

The present invention relates to method and apparatus for collecting very small biological samples for further processing.

### BACKGROUND OF THE INVENTION

Microfluidics is directed to the science of the flow of small amounts of liquid in very small liquid conduits. The liquids often flow in along flow paths that are micro-meters in size or less. In addition there are a variety of other dimensions that are of interest in modern science such as nano-fluidics and milli-fluidics. In very small areas such as the areas defined on a micro, nano, or milli scale, a liquid such as blood behaves differently than in a larger area.

With the advent of micro and nano technology a number of technologies are emerging that will manage micro, nano, and milli scale fluid flows. However, there are still many challenges involved in the flow of liquids in small areas.

Many microfluidic devices are defined by the fact that they include one or more flow paths or channels that are 1 mm or less in dimension. A number of different fluids are use in microfluidic devices such as protein or antibody solutions, buffers, bacterial cell suspensions, and whole blood samples.

To obtain blood samples for use in a microfluidic flow devices a lancet may be used. However, the lancet device is not part of the microfluidic device.

As such, a method and apparatus is presented for extracting and managing the flow of liquids in small areas for analysis.

### SUMMARY OF THE INVENTION

In view of the foregoing considerations, Point of Care (FOC) instruments are being developed, which carry out testing by the bedside or in the physician's office.

In one embodiment, a method and apparatus are disclosed for managing whole blood. Specifically, particles of the blood are captured, while the remaining fluid portion of the blood is directed and collected for further processing.

### BRIEF DESCRIPTION OF THE DRAWINGS

The accompanying drawings, which are incorporated into and form a part of the specification, illustrate one or more embodiments of the present invention and, together with the description, serve to explain the principles of the invention. The drawings are only for the purpose of illustrating one or more embodiments of the invention and are not to be construed as limiting the invention. In the drawings:

FIG. 1 displays a first perspective view of a sample collection device implemented in accordance with the teachings of the present invention.

FIG. 2 displays a second perspective view of a sample collection device implemented in accordance with the teachings of the present invention.

FIG. 3 displays a second embodiment of a sample collection device implemented in accordance with the teachings of the present invention.

### DETAILED DESCRIPTION

For the purposes of promoting an understanding of the principles and operation of the invention, reference will now be made to the embodiments illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to those skilled in the art to which the invention pertains.

Described herein are embodiments pertaining to fluid collection devices that provide for the procurement of a fluid sample, such as a biological fluid, and separating out debris from the fluid sample, without the need for increased gravity, such as through the use of a centrifuge.

The term “debris” as used herein in the context of fluid that is procured for sampling and analysis typically refers to particles in the fluid sample that have a density different than the fluid being collected. The particles typically, but not necessarily, have a density that is higher than the fluid. Debris includes, but is not limited to, living or dead cells or tissues, or fragments thereof, in the procured fluid sample. Fluids sampled typically refer to whole blood, but may also include, urine, semen, sweat, saliva, tears, mucus, tissue homogenates, and the like. In the case of blood, reference to “debris” is meant to also include interstitial fluid and/or intracellular fluid, as will be explained in further detail herein.

As used herein, the term “cleanse” (or other verb forms thereof), with respect to the fluid treatment refers to the separation of debris from the fluid that may be collected for further analysis. Cleansing does not necessarily involve complete separation, but rather, reduction in the amount of debris in the fluid sample.

Alternatively, the particles separated out from the fluid may also be analysed. For example, if the fluid is blood, blood cells may be separated out and counted to provide for a hemocrit analysis.

Certain embodiments pertain to simple to use devices that include a component that assists in accessing the fluid sample. In a specific embodiment, the component is a lancet that makes a micropuncture in the skin of a subject to allow for the flow of a small amount (e.g. less than a milliliter) of blood. Upon procurement of the blood sample, blood sample is then subjected to structural features of the device that encourage the separation of debris from the fluid to enable the collection of a cleansed fluid sample.

In clinical chemistry, the identification of the composition of a person's blood is used as an important diagnostic tool. Blood is primarily plasma, but also includes three major types of cells. Plasma comprises approximately sixty percent to seventy percent of a human blood sample, while approximately thirty to forty percent of the sample is cellular. Plasma within the sample is more than ninety percent water, with the remainder consisting of proteins, lipids, salts and the like. The three major blood cell types are red blood cells (RBCs), white blood cells (WBCs) and platelets.

Extracellular fluid is typically defined as body fluid outside of cells. The fluid found inside the cells is known as intracellular fluid. The cytosol or intracellular fluid is the liquid found inside of cells. In some animals, including mammals, the extracellular fluid can be divided into two major subcomponents, interstitial fluid and blood plasma. The extracellular fluid also includes the trans-cellular fluid, which is the portion of the total body water contained within epithelial lined spaces. The interstitial fluid is a solution that bathes and surrounds the cells of multicellular animals. The interstitial fluid is found in the interstitial spaces, also known as the tissue spaces.

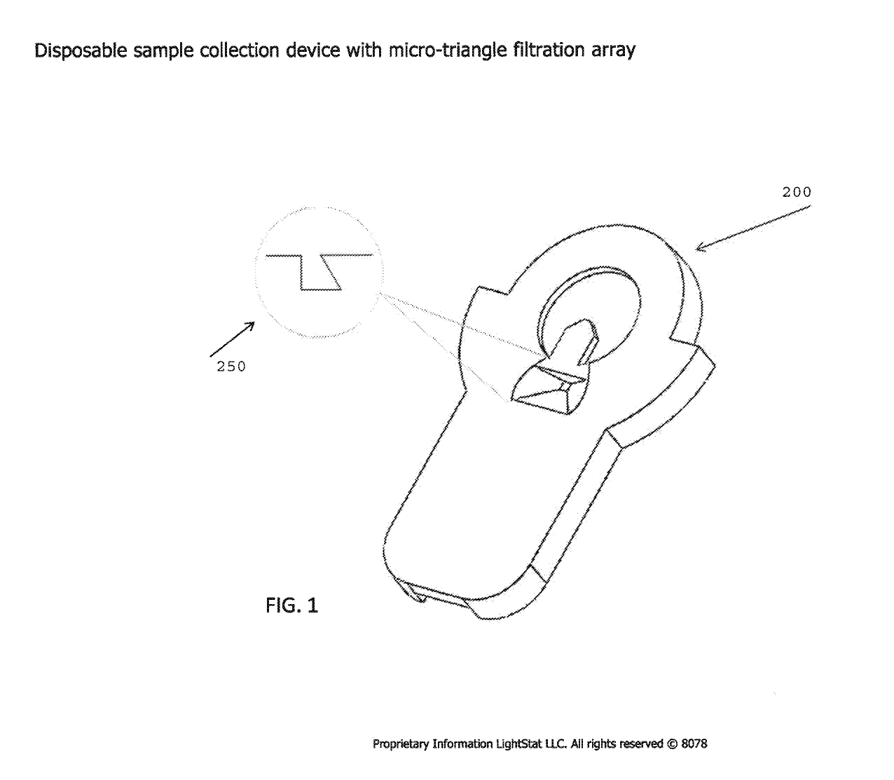

FIG. 1 displays a first perspective view of a sample collection device implemented in accordance with the teachings of the present invention. The sample collection device is shown as 100 with the debris container identified as 110.

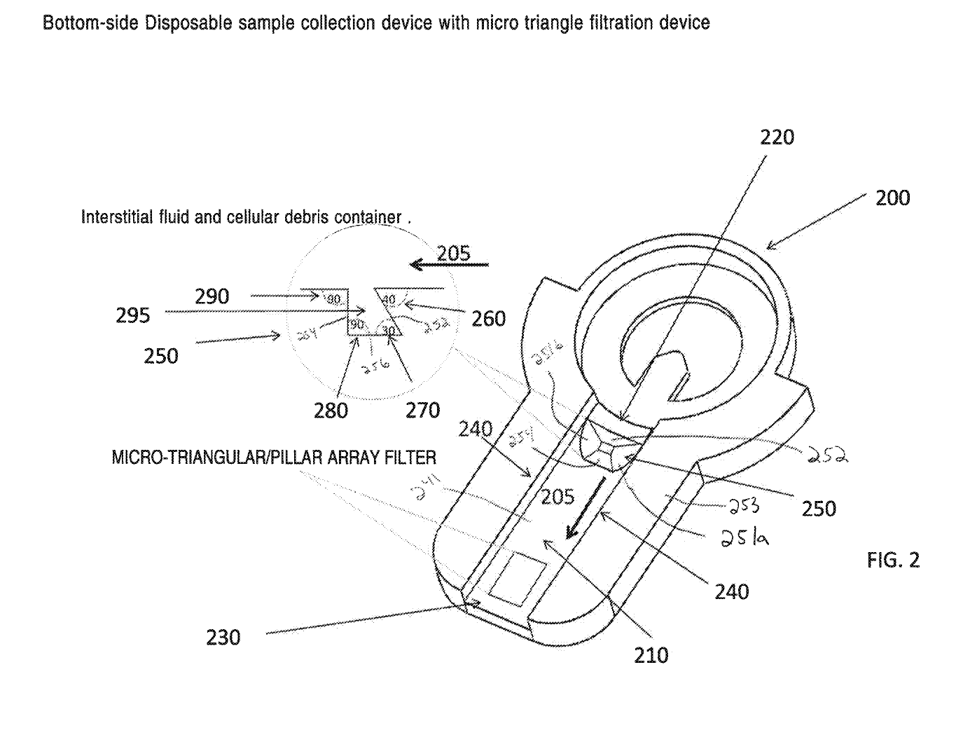

FIG. 2 displays a second perspective view of a sample collection device implemented in accordance with the teachings of the present invention. FIG. 2 displays a collection device including a debris container. The disposable collection device is shown as 200. A channel 210 is defined in a housing 253 of the device 200 by sidewalls 240 and a bottom wall 241. An inlet to the channel 220 and an outlet to the channel 230 are also shown. A liquid such as whole blood flows through the channel along a flow path 205. A debris container 250 is shown within the channel 210.

The debris container 250 includes an entry wall 252, opposing side walls 251a,b, and back wall 254. The debris container is shown adjacent to the channel 210. Typically, the container space 295 is below the level of the channel 210, but its walls are flush with the channel 210.

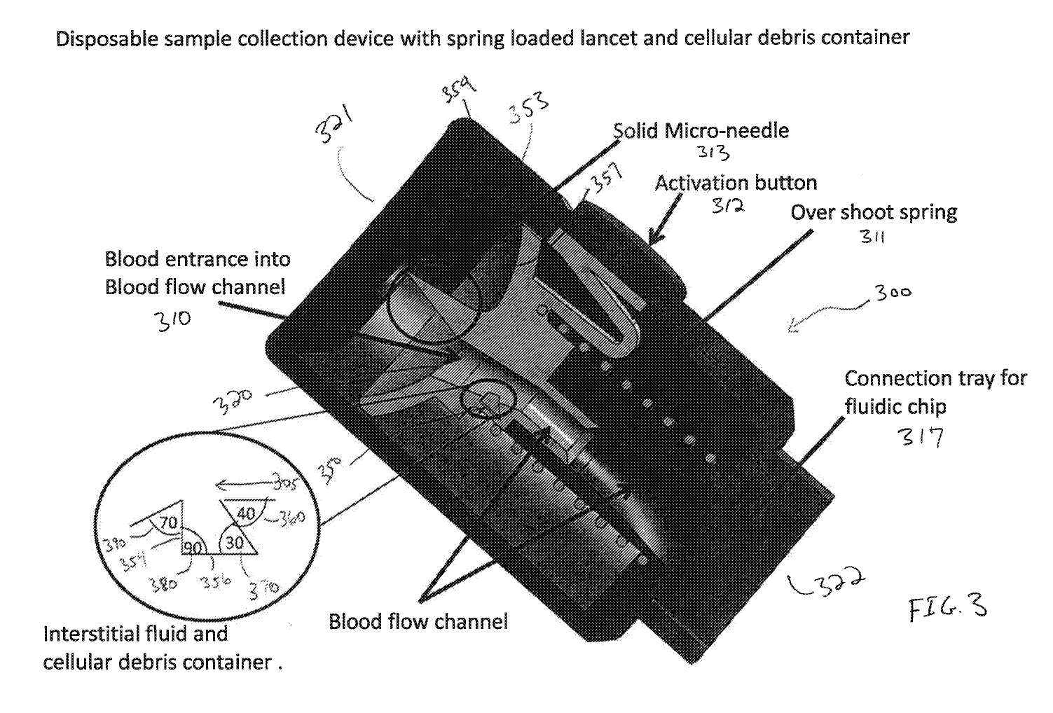

A magnified cross-section of the debris container 250 is shown. The container includes an entry angle 260 that involves an angle that is less than 90 degrees (shown in the Figure for exemplary purposes only as a 40 degree angle) to serve as an over-hang for the container and trap the interstitial and cellular debris. A first bottom angle 270 is angled at an acute angle (shown in the Figure for exemplary purposes only as 30 degrees) to also trap the interstitial and cellular debris. The second bottom angle 280 is shown as a 90 degree angle to provide a perpendicular surface to the flow path 205 when combined with the exit angle 290 Which is also defined with a 90 degree angle. Those skilled in the art in view of the teachings herein would appreciate that angles 280 and 290 might deviate from 90 degrees. What is important is that the flow of fluid over the container allows for entry of debris into the container. For example, the 90 degree or orthogonal angle has been found to allow entry of debris, but as will be discussed with respect to FIG. 3, the angle 290 can be less than 90 degrees (e.g. 70 degrees). Use of the term “about” in reference to an angle of the container is intended to mean the specified angled and up to a 15 degree variance greater or lower than the specified angle. The micro-triangular/pillar array identified in the channel 210 is designed for the separation of blood cells from the plasma following the cleansing that occurs in the container 250.

In an alternative embodiment, the container space is below the channel with a slanted “ramp” in which the deeper end is at the far end of the container in the direction of the flow. Naturally, to obtain this configuration the first entry angle and first bottom angle would need to be greater than 90 degrees.

In one embodiment, during operations, blood is introduced into the flow path 205. The initial drops of blood such as blood acquired from a skin puncture will include debris among other components. The debris can interfere with the analysis of components in the blood thereby providing inaccurate or false readings in later process steps. Thus, it is advantageous to capture these items and separate them from the sample. The debris container 250 captures these items in the container 295 space of the debris container 250.

FIG. 3 displays a cross-sectional view of a second embodiment of the sample collection device 300 including an overshot spring loaded lancet and debris container. The device 300 includes a housing 353 that has a distal end 321 and a proximal end 322. Disposed within the housing is lancet 313, which is shown as a solid micro-needle. A drive component 311 (shown as a spring) is operatively coupled with the lancet 313. The actuator 312 is configured to release the lancet 313, whereby the drive component 311 directs movement of the lancet 313 upon depressing the actuator 312. Based on the teachings herein, it will be appreciated that other drive mechanisms may be implemented, including, but not limited to, gas powered devices, hydraulic device, or even micro-motors.

Upon actuation of the lancet 313, a fluid such as whole blood is accessed from the subject (such as a human or other animal subject) and fluid is directed to the flow channel 310 at the channel inlet 320. In typical operation, a subject places their finger at the distal end 321 and the drive component 311 is actuated by the actuator 312.

Upon entry into the channel 310, the fluid encounters the debris container 350. Similar to that shown in FIG. 2, a magnified cross-section of the container 350 is shown that includes an entry angle 360 that involves an angle that is less than 90 degrees (shown in the Figure for exemplary purposes only as a 40 degree angle) to serve as an over-hang for the container and trap the debris in the fluid. A first bottom angle 370 is angled at an acute angle (shown in the Figure for exemplary purposes only as 30 degrees) to also trap the interstitial and cellular debris. The second bottom angle 380 is shown as a 90 degree angle to provide a perpendicular surface to the flow path 205 when combined with the exit angle 390 which is shown with a 70 degree angle.

Further to that described above with respect to the angles of the containers 250 and 350, the entry angle 260 or 360 is typically less than about ninety degrees. In a specific embodiment, the entry angle 260 or 360 is from about 15 degrees to 60 degrees. More specifically, entry angle 260 or 360 ranges from about 25 degrees to about 45 degrees. Further still, entry angles 260 or 360 are 40 degrees or about 40 degrees.

In addition, the first bottom angle 270 or 370 is one that is less than about 90 degrees. In a specific embodiment, bottom angles 270 or 370 range from about 15 degrees to 60 degrees. In a more specific embodiment, bottom angles 270 or 370 range from about 20 degrees to 40 degrees. More specifically, the bottom angle 270 or 370 is 30 degrees or about 30 degrees.

With respect to the second bottom angles 280 or 380, these angles may range from about 80 degrees to about 110 degrees. In a specific embodiment, second bottom angles 280 or 380 are 90 degrees or about 90 degrees.

With respect to exit angles 290 or 390, these angles typically range from about 60 degrees to 105 degrees. In a specific embodiment, the exit angles 290 or 390 are 70 degrees or about 70 degrees. In another specific embodiment, the exit angles 290 or 390 are 90 degrees or about 90 degrees.

The container 250 or 350 includes a predetermined, minimal volume that is intended to capture the first drop of blood which contains cell debris and interstitial fluid, or even intracellular fluid typically from rupturing of cells during lancing. The presence of these elements in the fluid flowing to the reaction area can cause interference in the reaction process, either in blocking the micro-channels (debris) or corrupting the actual chemical determination of the required analyte. A following blood volume also tops-off the container, suppressing the interstitial and cellular debris contained in the container 295 space and allowing uncontaminated blood such as the remaining drops of blood acquired from a puncture to flow along the flow path.

The container volume is designed such that once filled with debris, interstitial fluid, or intracellular fluid so that the subsequent drops of blood continue towards the separation area (such as the micro-triangular/pillar array filter shown in FIG. 2, which is the topic of a related application) where the blood cells are separated from the plasma which is used for the actual determination. The cleansing process followed by cell separation is intended to purify the sample flowing into the reaction area to pure plasma. Extracellular fluid will only be found in the first drop of blood collected due to the puncture with the lance. All subsequent drops will not contain debris or extracellular fluid

The embodiment 300 includes a connector 317 configured at the proximal end 322. The connector is adapted to allow for fluid communication with the flow channel 310 to carry the cleansed fluid to another device for analysis.

It is important to an understanding of the present invention to note that all technical and scientific terms used herein, unless defined herein, are intended to have the same meaning as commonly understood by one of ordinary skill in the art. The techniques employed herein are also those that are known to one of ordinary skill in the art, unless stated otherwise. For purposes of more clearly facilitating an understanding the invention as disclosed and claimed herein, the following definitions are provided.

It should be borne in mind that all patents, patent applications, patent publications, technical publications, scientific publications, and other references referenced herein and in the accompanying appendices are hereby incorporated by reference in this application to the extent not inconsistent with the teachings herein.

While a number of embodiments of the present invention have been shown and described herein in the present context, such embodiments are provided by way of example only, and not of limitation. Numerous variations, changes and substitutions will occur to those of skill in the art without materially departing from the invention herein. For example, the present invention need not be limited to best mode disclosed herein, since other applications can equally benefit from the teachings of the present invention. Also, in the claims, means-plus-function and step-plus-function clauses are intended to cover the structures and acts, respectively, described herein as performing the recited function and not only structural equivalents or act equivalents, but also equivalent structures or equivalent acts, respectively. Accordingly, all such modifications are intended to be included within the scope of this invention as defined in the following claims, in accordance with relevant law as to their interpretation.

What is claimed is:

1. A microfluidic sample collection device, comprising:
a channel including a flow path, the channel further comprising container submerged below a flow path in the channel.

2. A microfluidic sample collection device, as set forth in claim 1, wherein said container comprises an entry wall, opposing side walls, and a back wall opposite said entry wall.

3. A microfluidic sample collection device, as set forth in claim 1, wherein the container further comprises an entry angle, a first bottom angle, a second bottom angle, and an exit angle proportioned to trap fluid along the flow path in the channel.

4. A microfluidic sample collection device, as set forth in claim 2, wherein said entry angle is less than ninety degrees.

5. A microfluidic sample collection device, as set forth in claim 3, wherein said entry angle ranges from about 15 degrees to 60 degrees.

6. A microfluidic sample collection device, as set forth in claim 3, wherein said entry angle ranges from about 25 degrees to 45 degrees.

7. A microfluidic sample collection device, as set forth in claim 3, wherein said entry angle is 40 degrees or about 40 degrees.

8. A microfluidic sample collection device, as set forth in claim 3, wherein said first bottom angle is less than 90 degrees.

9. A microfluidic sample collection device, as set forth in claim 3, wherein said first bottom angle ranges from about 15 degrees to about 60 degrees.

10. A microfluidic sample collection device, as set forth in claim 3, wherein said first bottom angle ranges from about 20 degrees to about 40 degrees.

11. A microfluidic sample collection device, as set forth in claim 3, wherein said first bottom angle is 30 degrees or about 30 degrees.

12. A microfluidic sample collection device, as set forth in claim 3, wherein said second bottom angle ranges from about 80 degrees to about 110 degrees.

13. A microfluidic sample collection device, as set forth in claim 3, wherein said second bottom angle is 90 degrees or about 90 degrees.

14. A microfluidic sample collection device, as set forth in claim 3, wherein said exit angle ranges from about 70 degrees to 105 degrees.

15. A microfluidic sample collection device, as set forth in claim , wherein said exit angle is 70 degrees or about 70 degrees.

16. A microfluidic sample collection device, as set forth in claim 3, wherein said exit angle is 90 degrees or about 90 degrees.

17. A microfluidic sample collection device, as set forth in claim 1, wherein the container is parallel to the channel.

18. A microfluidic sample collection device as set forth in claim 1, wherein said device comprises a housing into which said channel is defined.

19. A microfluidic sample collection device, as set forth in claim 1, wherein said channel comprises channel sidewalls and a channel floor, wherein the container is set forth below the channel floor.

20. A microfluidic sample collection device, as set forth in claim 1, further comprising a lancet disposed on the housing.

21. A microfluidic sample collection device, as set forth in claim 20, wherein the lancet is adjacent to the channel such that fluid accessed by the lancet enters said flow path.

22. A microfluidic sample collection device, as set forth in claim 20, further comprising a drive mechanism and an actuator, wherein upon actuation said drive mechanism moves said lancet.

23. A microfluidic sample collection device comprising:
a housing having a distal end and a proximal end;
a channel defined in said housing such that fluid entering said device travels in a flow path from said distal end to said proximal end;
a container adjacent to said channel and in fluid. communication therewith; and
a lancet disposed in said housing.

24. A microfluidic sample collection device, as set forth in claim 23, further comprising a drive mechanism for moving said lancet.

25. A microfluidic sample collection device, as set forth in claim 24, wherein said drive mechanism is a spring, pneumatic device, or hydraulic device that is coupled to said lancet.

26. A microfluidic sample collection device, as set forth in claim 24, further comprising an actuator for actuating said drive mechanism.

27. A microfluidic sample collection device, as set forth in claim 23, further comprising a fluid tray connector proximate to said proximal end.

28. A microfluidic sample collection device, as set forth in claim 23, wherein the container further comprises an entry angle, a first bottom angle, a second bottom angle, and an exit angle proportioned to trap fluid along the flow path in the channel.

29. A microfluidic sample collection device, as set forth in claim 28, wherein said entry angle is about 40 degrees, said first bottom angle is about 30 degrees, said second bottom angle is about 90 degrees, and said exit angle is about 90 degrees.

30. A method of obtaining fluid sample from a subject utilizing a microfluidic sample collection device according to claim 23, wherein said fluid sample comprises a reduction in interstitial and cellular debris; said method comprising:
accessing a raw fluid sample at said distal end via application of said lancet to said subject;
allowing said raw fluid sample to flow through said channel such that said raw sample interacts with said container; thereby producing a cleansed fluid sample downstream said container; and
collecting said cleansed fluid sample or subjecting said cleansed fluid sample to analysis.

31. A method according to claim 30, wherein said collecting step occurs at said proximal end.

32. A microfluidic sample collection device of claim 23, wherein said lancet is a microneedle.
